(19) United States
(12) Patent Application Publication
(10) Pub. No.: US 2008/0160599 A1
(43) Pub. Date: Jul. 3, 2008

(54) Unit For Processing Microbiological Samples

(75) Inventors: Klaus Weber-Matthiesen, Achterwehr (DE); Peter Ivan Sygall, Riethoven (NL); Johannes Jansen, Schwabisch Hall (DE)

Correspondence Address: DUANE MORRIS, LLP;IP DEPARTMENT, 30 SOUTH 17TH STREET, PHILADELPHIA, PA, 19103-4196, US

(73) Assignees: Klaus Weber-Matthiesen; Bioplan Consulting GmbH

(21) Appl. No.: 11/574,601

(22) PCT Filed: Sep. 2, 2005
(86) PCT No.: PCT/EP2005/009441

§ 371 Date: Sep. 13, 2007

(30) Foreign Application Priority Data
Sep. 3, 2004 (DE) 10 2004 043 399.2

(51) Int. Cl. C12M 1/00

(52) U.S. Cl. 435/286.2; 435/307.1; 435/303.1

(57) ABSTRACT

A unit for processing microbiological samples has a conveyor for transporting samples, for example in microtitre plates. At a processing station, samples for processing are transferred in their sample containers from the transport device to a processing device, which can have several processing areas, of which only one is ever in a processing position. A manipulating device is associated with the processing position. A magazine dispenses sample containers for processing. An incubator accumulates and discharges sample containers using transport and storage arrangements with coupleable conveyors on multiple levels.

The invention relates to an automatic system for processing biological samples or to one that can be automated.

It is known that biological samples disposed in microtubes are processed in a special manner. Said processing consists of drawing certain quantities of liquid from liquid samples, introducing reagents, verifying various parameters and so forth. Since large quantities of samples must be processed frequently, microtubes are often kept in receptacles which may have the form of microtitre plates. It is also already known that part of the microtitre plates may be transported by means of manipulating devices. It is possible to provide microtitre plates with machine-readable labels, and save the positional data of a specific sample in a special microtitre plate in an electronic memory.

A processing device for biological samples is known from the European Patent Application 1 348 485.

The invention is based on the task of creating a possibility of employing very different methods of processing biological samples in a large number and, if necessary, also under clean-room conditions, according to various criteria and in particular when these criteria first arise or change during the processing stage.

To solve this task, the invention proposes a unit for automated processing of biological samples, with the features mentioned in Claim 1.

The unit comprises a transport device that can he operated by a controlled drive to transport the sample container. The container can also involve cavities like a microtitre plate or other types of sample containers, e.g., wells. The unit further contains at least one processing position to which the sample to be processed is conveyed. It is processed with a processing device, for example a pipetting device. This processing device is positioned by the manipulating device in such a manner that the processing action can occur with said device, at this point, opposite the processing position.

This type of distribution of tasks has the advantage that individual devices or equipment—transport device, processing device and manipulating device work at the greatest extent independent of one another, so that their work can be controlled, checked, and influenced by a central control unit. The individual manipulating devices can be adapted to their special task. For example, the transport device is exclusively meant for transporting all the required sample containers and consumables. The task of the manipulating device is processing the sample container in the prescribed manner, with the help of processing devices.

To be able to provide empty sample containers in a further development according to the invention, the unit can feature a magazine for providing sample containers and equipment for removing the sample containers from the magazine and handing them over to the transport device. Also this manipulating device also works independent of the other manipulating devices; however, it can be controlled by the central control unit.

To be able to process samples which require a specific period of processing under specific conditions, the unit can feature an incubator in a further development according to the invention; said incubator can be used for accommodating samples in a defined environment.

To further develop the advantage mentioned at the beginning—the independence of transport and processing according to the invention—it can be provided according to the invention that the processing position is disposed outside the transport section of the transport device. Under the term “transport section” the section along which the sample containers that are connected with the transport device are transported is understood. “Outside this transport section” implies that the sample containers are displaced far enough apart that the transport device can continue operating. Therefore, the processing position can be disposed laterally on each of the two sides of the transport section, but also above or below the transport section.

For the delivery of a sample container from the transport device to the processing position, the unit can feature transfer equipment according to the invention. This transfer equipment can also serve the purpose of transferring the sample container from the processing position back to the transport device, after processing. Depending on the circumstances of an individual case, a separate manipulating device can be provided as transfer equipment from the transport device to the processing position, and a second transfer equipment can be used for delivery of sample containers from the processing position to the transport device.

The transfer equipment for example can work in such a manner that when the transport device stops, delivery takes place. It is also possible that the transfer equipment is moved together with the transport device so that a changeover action can occur whilst the sample container is moved by the transport device.

In a further development of the invention, it can be provided that this transfer equipment operates likewise independent of the other devices, and in particular, independent of the manipulating device.

It was already mentioned that the actual processing of the biological sample occurs at the processing position. In so-doing, the sample container remains stationary at the processing position. The processing device is brought to this position by the manipulating device. In the case of sample containers that are very small in size, the manipulating device positions the processing device in such a manner that it meets the opening of the sample container. In a further development of the invention it is proposed that the manipulating device is not only designed for mechanical coupling to the processing device, but also for functional coupling. For example, a power supply can also occur by establishing a plug connection or a similar contact via a mechanical coupling between the manipulating device and the processing device. Even a mechanical drive can be coupled between the manipulating device and processing device in case the processing device should also execute a mechanical task. It is also considerable that negative air pressure or excess pressure media can be connected by coupling it to the processing device. In this manner it is ensured or provided among others that the processing device is only then ready for processing, when it is coupled to the manipulating device. This measure also serves the purpose of using as few cable and hose connections as possible on the processing device. Hoses and cables moved around in a clean room are a major source of contamination.

It is also possible to couple media (culture solutions), water, cleaning solutions and a drainage line via the manipulating device, which helps avoid hoses on the processing devices.

The processing device is disposed in a waiting station when it is not in operation. This waiting station is disposed outside the processing area, in such a manner that it does not conflict with the transport of the sample container and the delivery of the sample container at the processing position.

In a further development of the invention it can be provided that the waiting station is designed in such a manner that it not only holds the processing device ready for later processing at the station, but also cleans the device during working breaks. During the periods in which a special processing device is not in use, it can be cleaned so that the waiting time of the processing device can be exploited. This improves the entire throughput of the unit. In addition, a functional coupling can also be provided between the processing device and depositing station. This involves a functional coupling with a different task, in this manner, the power supply to the processing device should be achieved when depositing and when controlling the self-cleaning action during processing breaks.

Since many various processes must be frequently carried out with biological samples, in a further development of the invention the unit can feature several independently working processing devices which respectively feature their own waiting station. The controls of the unit control the manipulating device such that it selects the required processing device for the respective task and brings it to the processing position.

For processes with substantial time requirements for cleaning the processing devices, for example sterilization, after each individual processing, it can be advantageous to provide several similar processing devices. This is possible by separating the manipulating device from processing devices on the basis of the present invention.

By separating the transport area, processing area, manipulating device, and processing devices according to the invention, it is made possible when handling series processes with high cleaning requirement between individual actions that handling two processes can take place alternately;

for example 1-2-1-2-1-3-1-4-1-2-1-2, wherein 1 represents a series process with high cleaning requirement.

A processing device, of course, can be designed so that it processes numerous sample containers, for example for all microtubes of a microtitre plate. The processing devices can also be designed so that selecting the sample container which is located at the processing position is possible, so that the processing task takes place appropriately.

In a further development of the invention it can be provided that the waiting positions of the processing devices and/or of the processing position are disposed such that the manipulating device can select and approach the positions of the processing devices by executing a linear motion along a single degree of freedom. For example, with a manipulating device movable along a linear line, also the waiting positions can be disposed along the linear line. However, if a manipulating device that is rotatable about a fixed axis is used, then it is reasonable to dispose the waiting positions of the processing devices along a circular path.

It is likewise reasonable according to a further feature of the invention when a degree of freedom of the manipulating device comprises a motion parallel to the transport direction of the transport device.

To further improve the efficiency of the unit, it can be provided that the processing position itself is movable, preferably in a direction parallel to the transport direction of the transport device. This is especially useful when with several processing devices the area in which the waiting positions of the processing devices are disposed is somewhat larger and a processing device requires a hose connection. Then the processing position can be moved nearer to the waiting position of the processing device. This is especially advantageous when processing in a clean room, since, through the uncritical motion of the processing place, critical motion of supply hoses above the product level is avoided.

When hoses and cables can be omitted due to media supply via the manipulating device, it is then on the other hand also possible to dispose the processing stations in a stationary setup. Then they do not need to have processing tools with restricted mobility to be railed.

When the processing position is movable, for example, with the help of a carriage, then in a further development according to the invention, the carriage can also feature several processing stations of which always only one can be connected with a processing device in a processing position. In this manner, hand-over actions can take place whilst processing continues.

The transport device that moves the sample container is preferably a revolving transport device in which the sample containers move along a closed path. It is particularly reasonable when this closed transport path features two parallel sections and two connection return pulleys. The transport path therefore corresponds to an oval shape. The manipulating device can for example be located adjacent to a linear section. Due to spatial reasons, however, it is preferred to have the manipulating device and/or the processing device and/or the processing position disposed within the return pulley. Relatively many waiting positions can be disposed there, which can be controlled by the manipulating device without covering a great distance.

The processing position can be advantageously disposed between the transport section and the waiting positions of the processing devices.

According to the invention, microtitre plates can be provided as sample containers.

The transport of sample containers can take place for example in such a manner that the transport device features receptacles for the microtitre plates at regular intervals; a receptacle is provided for a microtitre plate respectively. The transfer equipment can be designed in such a manner that if hands over the microtitre plate or the sample container or several sample containers to the processing position. It is likewise possible and proposed by the invention that the transfer equipment transfers the receptacle with the sample container to the processing position.

It was previously mentioned that the unit can feature a magazine for storing empty sample containers. Magazines of this type are frequently designed as carousel towers which can be rotated by a drive, wherein the delivery occurs along a vertical row in one position.

The invention now instead proposes that the magazine features at least a linear line that leads to the front side for a row of sample containers to be disposed serially, preferably numerous rails disposed one above the other. In this manner a large number of sample containers may be accommodated in a small space. Preferably, several such arrangements of rails are disposed alongside each other.

In a further development of the invention, it can be provided that the rails are made in such a manner that the containers are loaded in the direction of the front side against a stopper. In this manner, it can be ensured at the delivery points that on the front side, a sample container is always at disposal, provided that a sample container is still available in the guideway. It can be attained there by means of a manipulating device.

Inlet from the front can occur by means of driven rollers on which the containers are placed.

A further possibility of loading the sample containers can comprise the provision of revolving belts, for example, two parallel revolving belts on which the container lies. The belts can then be driven by motors.

For the feed process, it can be provided that a slip prevails between the belt and rollers, or rollers and the containers. However, it is likewise possible and within the scope of the invention that the slip occurs between the belts or rollers and the drive.

A further possibility comprises the fact that a loading process is generated by means of a ramp configuration of the conveyors. Thereby it can be provided that the conveyors are roller conveyors, in that the sample containers lie on rollers and are loaded against the stopper on the front side, by means of their own weight, if the roller conveyors on this front side feature a downward inclination a drive becomes unnecessary for the magazine.

A further possibility involves a conveyor belt coated with a smooth-slide material so that the sample containers are likewise fed forward and slide along the conveyor line under gravity.

The manipulating device removes the first sample container from each row respectively, whilst the others slide or roll in place. Through the plurality of discs made of roller conveyors disposed one above the other, magazines can be built besides each other, which can accommodate a large number of microtitre plates for example in a congested space. Thereby it can be provided that the roller conveyors feature a top guide for the sample containers, in particular for the microtitre plates. It can be provided that the distance between the top side of the rollers and the bottom side of the respective top guide is smaller than the sum of the height of a removed lid and the lid-less bottom part of the sample container.

It is particularly useful to design the roller conveyors in such a manner that the top guide of the rollers is formed by the top roller conveyor respectively.

For the removal of sample containers, the manipulating device can be designed in such a manner that it lifts the front sample container above a stopper.

A second possibility is that if releases a stopper in front of the sample container and slides it into the gap opened horizontally during the transition of the container in front of the next sample container.

During the transition of the sample container from the roller conveyor with a downward slope, thus inclined to the horizontal plane, necessary for the transport device, a gap always occurs between the sample container disposed horizontally and the next sample container. This gap has the effect that severe stress occurs on the front fop edge on the following sample container. This can cause the lid to pop off. Furthermore, when loading roller conveyors with products, the products hit the stopper edge with full force at the delivery opening or hit a product already delivered. Even here the lid of the incoming product could pop off and block the guideway. Also when the front container is raised, the danger of the lid popping off always exists. The lid is prevented from popping off, since the room above the lid and below the top guide is dimensioned such that it is prevented from popping off.

A possible incubator for the unit according to the invention can be disposed such that, the delivery of a sample container in the incubator and from it outwards occurs in a linear and horizontal line. For this purpose, the incubator can feature an opening that is disposed at the same height as the transport device. The opening serves for entering and removing sample containers into and out of the incubator.

It features a size in height such that a sample container just fits through it. A manipulating device for entering and removing sample containers is disposed outside the incubators and can be moved along the opening. The opening is so long that all sample containers can be pushed directly into the receptacles available inside the incubator.

In a further development of the invention, it can be possible that the incubator is designed in such a manner that the sample container can be moved by means of the manipulating device provided inside the incubator. However, by which the drive is located outside the incubator, it can be coupled with the drive by means of shafts introduced through the walls and mounted on bearings. These bearings can be sealed properly. By disposing the drive outside the incubator, there is little danger of contamination. This is particularly important because clean-room conditions must prevail inside the incubator. Moreover, the possibility exists to sterilize the interior space by means of aggressive gases or chemicals.

The drive can be designed in such a manner that the sample container within the incubator is slowly moved in order to normalize the climatic conditions in the interior space.

To ensure that the conditions prevailing in the interior are disturbed only as little as possible when loading and unloading the incubator, it can feature an inlet sluice in a further development according to the invention so that it is only opened for the purpose of filling and removing the content. For example, this inlet sluice can be designed similar to a revolving door. Since the incubator can be open for a long period due to the plurality of sample containers to be disposed side by side, the door of the sluice can be divided according to the invention.

It is also possible for the unit to have an incubator that features a plurality of receptacles for sample containers according to the invention, which are disposed along a closed path and are transportable along said path, preferably in both directions. Under “sample containers” one can in this case also consider microtitre plates which in turn accommodate a plurality of microtubes or similar manipulating devices. The motion along a closed path means that each receptacle is brought to a point at which an opening is provided for charging and discharging the incubator by removing its contents. The closed path for example is formed in that the receptacles are attached to and in particular suspended on two revolving chains, belts or other means similar to a paternoster. In this manner, the sample containers can also be moved within the incubator, so that all sample containers are kept in the same room.

In a further development of the invention it can be provided that the chain, the shelf, or a similar item features multiple strands running perpendicular to it. This allows the height of the incubator to be reduced so that the interior takes on a rather cubical shape, with more uniform conditions.

In a further development of the invention the incubator can feature multiple levels which consist of revolving belts capable of being driven or feature such drives. At each level, parallel rows of sample containers may be accommodated.

In a further development of the invention, a transport segment can be provided, which can be moved perpendicularly and can also be stopped at any height or at least at the heights corresponding to some of the transport levels. In this manner, it is again possible to cope with one inlet opening, which may feature a length that stretches across the entire width of the incubator housing, however which only needs to be so high that a sample container can be pushed through it, from the outside. This can be raised or lowered to the corresponding level by the transport segment. The transport segment can accommodate a series of sample containers, which are located beside one another. Two-dimensional fields of sample containers are provided at said levels.

In yet a further development of the invention, a drive coupling can be provided between the transport segment and the revolving belt of the levels, which can be activated when the transport segment is at the height of the respective level. Then, through the movement of the transport segment, a series of sample containers can be transferred onto the level, wherein the level is advanced by one cycle during this transfer process. For this purpose, no active drives are required for the conveyor belts.

In yet another development of the invention, it can be provided that the incubator features a return segment, which, similar to the transport segment, can be moved perpendicularly and laterally and can stop at the height of the level. This return segment is used for receiving the sample container that is pushed through the levels and returned back to another level, from where it is brought back to the position where the transport segment is located.

A drive coupling can also be provided between the return segment and the revolving belt of the level. This can be activated when the heights and the coupling between the drive and the return segment at said level correspond. The return segment can preferably be driven in both directions, as the transport segment.

A manipulation device can be provided to feed the incubator, which may be disposed outside the incubator and can be moved along the inlet opening of the incubator. According to the invention the unit in a further development can feature a loading station for manual delivery or removal of sample containers to or from the transport device, in case a manual action is desired at a certain point or under rarely occurring conditions.

According to the invention, the unit can likewise feature an automatic ejection station for sample containers when they are no longer needed.

The unit can feature a device for removing the lids of sample containers directly prior to their delivery in the processing stations. To carry out special and rare further processing, individual additional analysis co-processing stations can be disposed along the transport section. The magazines discussed here and the incubator can also be used independent of the type of the unit described here, so that independent protection is claimed for them. Additional features, details, and preferences of the invention result from the claims and abstract, both of whose wordings refer to the content of the description, which includes the following description of a preferred embodiment of the invention and the drawing as well.

The figures illustrate the following:

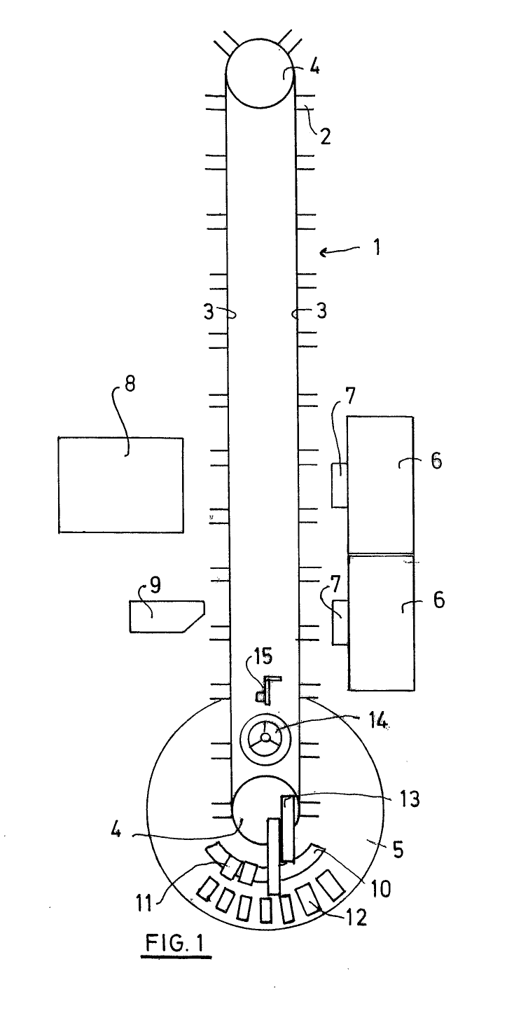

FIG. 1 shows a schematic overview of a unit for processing and handling biological samples according to the invention;

FIG. 2 shows an illustration of the spatial allocation of the manipulating device, transport section, processing position, and handling device;

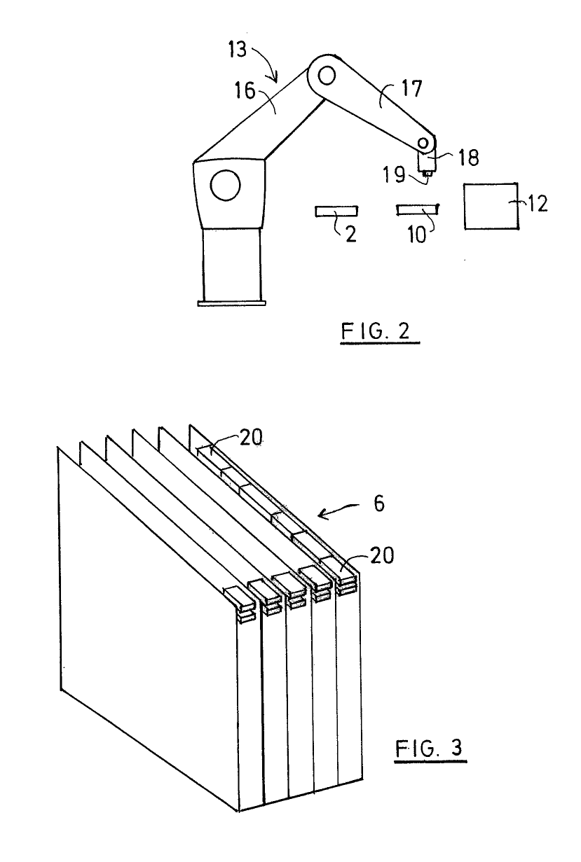

FIG. 3 shows a schematic view of a magazine for storing sample containers;

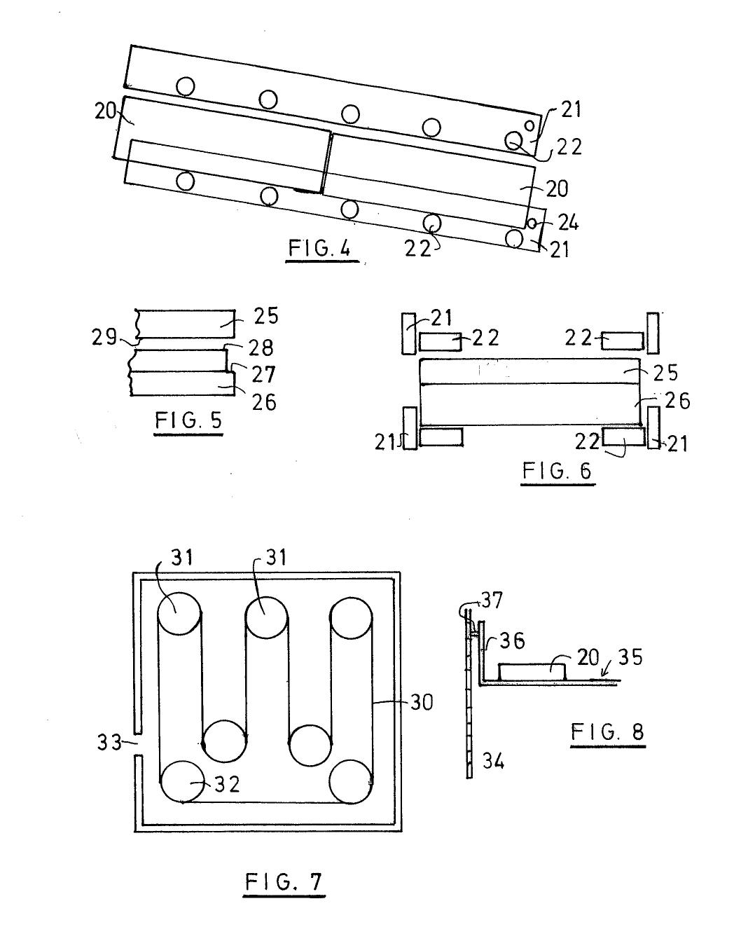

FIG. 4 a schematic illustration of a compartment of the magazine;

FIG. 5 a simplified illustration of containers for receiving samples;

FIG. 6 a schematic illustration of a compartment of the magazine;

FIG. 7 a schematic illustration of an incubator;

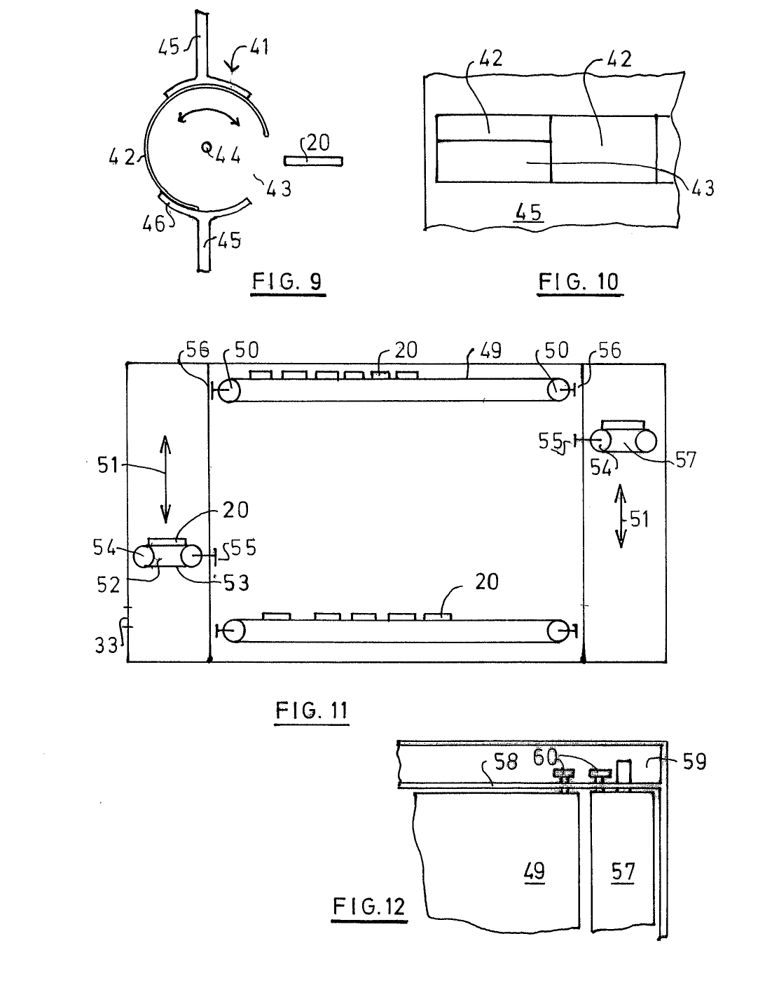

FIG. 8 shows schematic disposition of a receptacle for the incubator according to FIG. 7;

FIG. 9 shows a schematic side view of a sluice for incubator opening;

FIG. 10 shows a partial view of the incubator opening from the right, according to FIG. 9;

FIG. 11 shows a schematic disposition of a second incubator;

FIG. 12 shows a simplified section through the type of drive for the incubator according to FIG. 11;

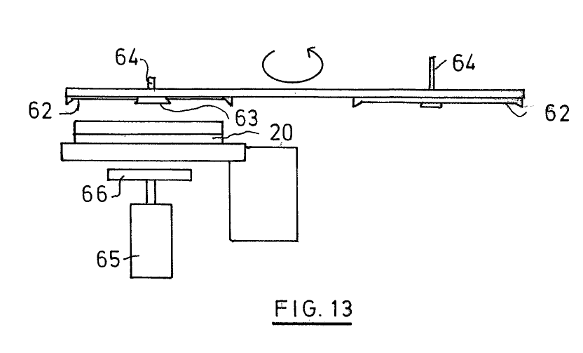

FIG. 13 shows the disposition of an apparatus for removing a lid from a sample container;

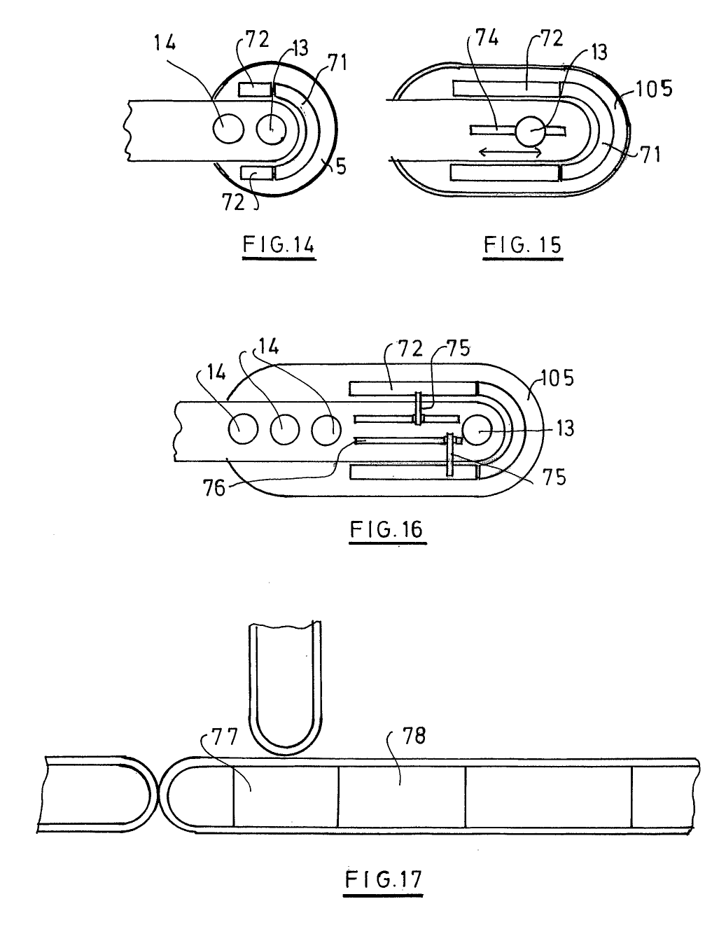

FIG. 14 shows the plan view of the processing end of a unit in a

simplified form;

FIG. 15 shows a plan corresponding to FIG. 14 for a modified embodiment;

FIG. 16 shows the plan of an embodiment once again modified in contrast to FIG. 15;

FIG. 17 shows a schematic arrangement of several revolving transport devices;

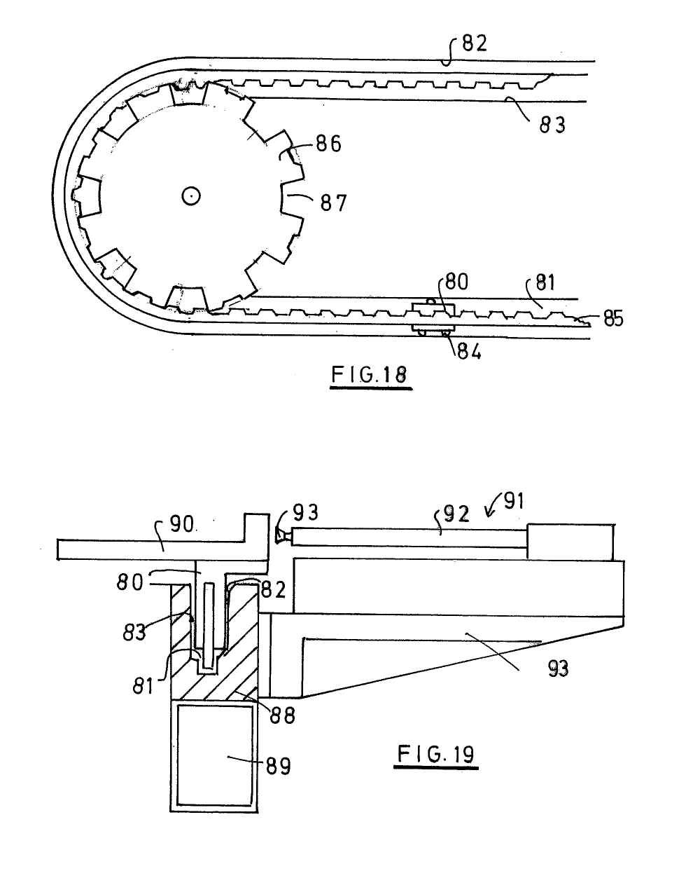

FIG. 18 shows a return arrangement at the end of a transport section, in a magnified scale;

FIG. 19 shows equipment for the delivery of receptacles onto the transport device;

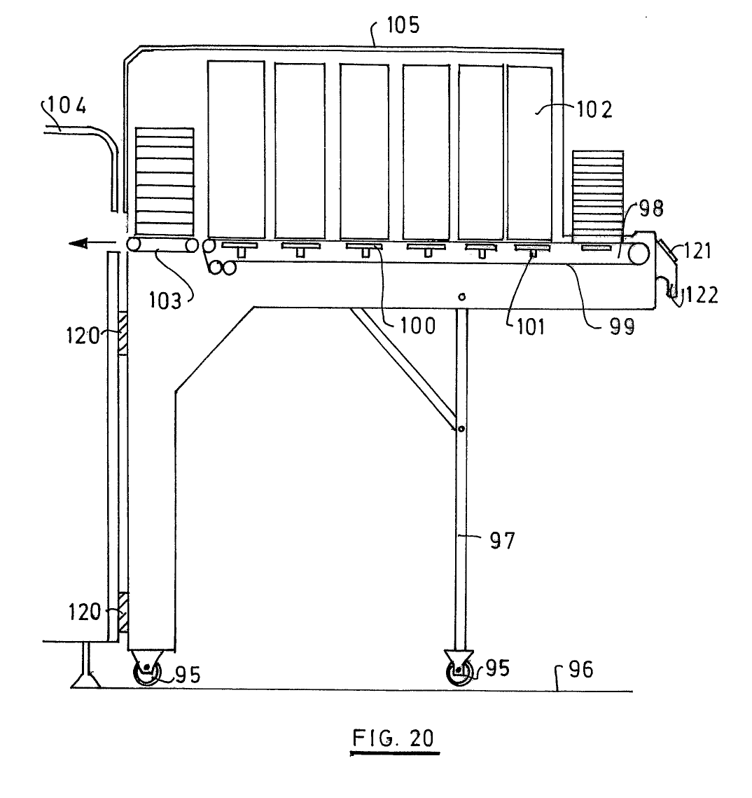

FIG. 20 shows the side view of equipment for the storage of consumables;

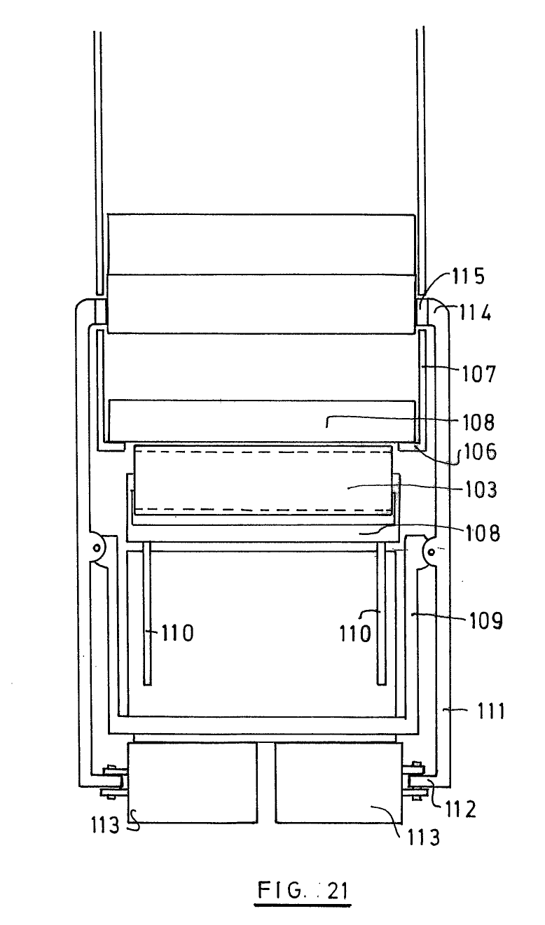

FIG. 21 shows a front view of the disposition of FIG. 20, from left in FIG. 20.

FIG. 1 shows an example of a unit according to the invention, in an overview illustration from top. The unit contains a transport device 1, for example which is made of a chain or a belt. This transport device is driven with the help of a drive (not depicted), wherein in principle, motion is possible in both directions, however, the motion in only one direction is preferred. The transport device drives a plurality of receptacles 2 for sample containers along a closed path, which feature two parallel linear sections 3, which are connected with one another by two return pulleys 4. When one considers the transport device as featuring a revolving chain, then the return pulleys 4 would be made of return gear wheels. It is nonetheless not necessary for the transport device to actually comprise a chain. A guide can be provided also, which features the shape depicted in FIG. 1, for example. The receptacles 2 are located at regular intervals and are driven by the transport device. The motion can be intermittent or continuous.

Biological samples are processed at the lower end of the transport device 1, as depicted in FIG. 1. A base 5 is located where different details of the unit are located; the details are yet to be explained.

Besides the linear running sections 3 of the transport device 1 in the depicted example, two magazines are disposed 6 for empty sample containers. Each magazine 6 is provided with a manipulating device 7, which takes a sample container, for example, a microtitre plate from the magazine 6 and places it in a receptacle 2. These sample containers are then brought by the transport device up to the processing station at the bottom in FIG. 1.

An incubator is disposed on the opposite side, in which a full sample container can wait for a certain period whilst the sample is being processed.

A transfer equipment 9 is used to feed the receptacles 2 on the transport device 1, which is not explained in detail.

On the machine base 5, the manipulating devices for processing the samples are at disposal. A carriage 10 is disposed on a circular guide outside the place occupied by the receptacles 2, which is restrictedly movable in both directions about the centre of the return pulley 4. Several receptacles 11 for microtitre plates are provided on the carriage 10. Likewise, several processing devices 12 are disposed outside the place occupied by the carriage 10 and the receptacles 11, where they feature their waiting position. The waiting positions of the processing devices 12 therefore lie on a circular guide that is disposed concentrically around the carriage 10 and around the return pulley 4.

In the return pulley itself, a manipulating device 13 is attached rotatably about the midpoint of the return pulley 4, which can be swivelled about the midpoint of the return pulley 4 and can execute other movements.

In the case of the depicted embodiment, the receptacles 2 are disposed outwardly the transport device 1, and the magazine 6 and the incubator 8 are also disposed outwardly. During the delivery of sample containers from the magazines 6 to the receptacles 2 and from the receptacles 2 to the incubator 8, the actual transport device does not disturb. With more complicated transfer equipment, also the receptacles 2 on the one hand and the incubator 8 and magazine 6 on the other hand could be disposed on different sides of the transport device.

As already mentioned, the receptacles 11 of the carriage 10 are disposed outwardly of the area of the receptacle 2 of the transport device, and the waiting positions of the processing devices 12 are again outward of the area of the carriage 10.

Besides the manipulating device 13, a carousel 14 is disposed, which can hold the lid of the sample container. A gripper 15 can remove the lid of the sample container approaching the processing station and place if on the carousel 14. After passing the processing station, the gripper 15 can again take the respective lid from the carousel 14 and place it on the sample container. The carousel 14 rotates at the same speed as that of the transport device 1. In this manner, one can assign each lid to the sample container, from which it was removed.

Disposition of the processing devices 12, the carriage 10 and the receptacles 2 of the transport device are once again illustrated in FIG. 2. FIG. 2 is to be understood very schematically. The processing devices 12 are disposed in their waiting positions outside the guide rail of the carriage 10. Several receptacles 11 for microtitre plates are provided on the carriage 10. The transport path of the receptacles 2 of the transport device 1 lies nearest to the midpoint of the processing station in which the manipulating device 13 is disposed. The manipulating device can not only rotate about the midpoint axis of the transport device 1 in the return pulley 4, but can also swing both arms 16 and 17. At the front end, once again a shorter arm 18 is attached in a manner allowing it to swing, on which a coupling device 19 is provided on the free end. With the coupling device 19, the front arm 18 of the manipulating device 13 couples a socket on the processing device 12. Then this manipulating device 12 is removed from its waiting position and positioned opposite a receptacle 11 on the carriage 10 and then is lowered so far that the processing device 12 can process the sample container in the receptacle 11 on the carriage 10. The sample container was transferred by the transfer equipment (not depicted) from a receptacle 2 of the transport device to the receptacle 11 on the carriage 10. The processing of a sample container occurs always in a receptacle 11 on the carriage 10, wherein always only one receptacle 11 is located in a processing position. This processing position does not need to feature a fixed place, but can change over a certain section of the motion of the carriage 10. With processing devices 12, which feature a hose connection or a line, the receptacle 11, on which the processing should occur, is moved closer to the corresponding device 12. Therefore, always only one processing position exists, even when several receptacles 11 filled with sample containers are available on the carriage 10.

Now regarding FIGS. 3 to 6 which depict a magazine 6 in magnified detail: the magazine 6 contains several plates beside one another, on guides disposed over one another, for the sample container 20. FIG. 3 shows this only schematically. Even partition walls do not need to be located between individual plates since if is sufficient to have grid-like structures for individual guides, in the individual guides, the sample containers 20 are disposed successively, wherein the first sample container 20 is prevented from sliding off, by means of a stopper, visible in FIG. 4. The rest sample containers 20 lie on the respective front container due to their weight. FIG. 3 shows such a magazine viewed from its front side. The front side is the respective side on which delivery should take place with a device 7.

FIG. 4 shows two sample containers 20 in a schematic side view, disposed in series in a guideway of a magazine. The guide rails are made as roller conveyors. On lateral wings 21 rollers 22 are rotatably mounted. The distance between two rollers 22 is chosen such that a sample container 20 in each position lies on several such rollers 22. As one can also derive from FIG. 4, the distance between the top side 23 of the sample container 20 and the rollers 22 of the next higher side rail 21 is small. The rollers 22 of the respectively next higher side rail 21 form not only a guide for the sample container available there in case the top most side rail 21 is not involved, but also a guide for the lower sample container 20.

As already mentioned, the front sample container 20 of each guideway is prevented from falling off the magazine or sliding out or roiling out by a stopper 24. To remove a sample container 20 from the magazine, the manipulating device must lift the sample container 20 at its front bottom edge far enough up that it goes over the stopper 24. This is possible since the space between the top side 23 of the sample container 20 and the roller 22 of the top side rail 21 is a bit wider than the distance of the top edge of the stopper 24 from the top edge of the roller 22 lying below it.

Should the front container 20 be lifted around its front side so that it approaches the horizontal plane, then its rear fop edge presses against the front top edge of the lid 25 of the following sample container 20. This poses the danger that the lid of this sample container 20 may pop off. The sample containers must however remain provided with the lid until they reach the processing station. Microtitre plates that are typically used as sample containers look as schematically shown in FIG. 5. The sample containers have a lid 25 and a lower part 26. The lower part 28 features a step 27, which features a certain distance from the top edge 28 of the lower part. The lid 25 is made such that it encompasses the remaining strip of the lower part above the step 27 of the lower part 26. In a closed state, the bottom edge 23 of the lid 25 rests on the step 27. To be able to lift off the lid 25 from the lower part 28, the lid 25 must therefore be raised by the distance between the top edge 28 and the step 27. To prevent the lid from popping off inadvertently, the distance between two conveyors 21 disposed above one another is determined such that the distance between the top side 23 of the lid 25 and the rollers 22 above it is smaller than the distance of the top edge 28 from the step 27. This allows such a magazine to be used in this application case, where, due to the transition between an inclined guideway and a horizontal position, the danger that a lid may pop off exists.

FIG. 6 shows a view of a sample container in a guideway with the lid and lower part, viewed from the front. One sees here that the rollers 22 on the side rail 21 only support the sample container 20 along its edges, so that the middle area remains free. A manipulating device can be applied here.

FIG. 7 shows an incubator in a very simplified form, as it can be used in a unit for processing biological samples. In the housing with an insulating wall, a revolving means of transport 30 is disposed, which is guided by three guide pulleys 31 disposed in the top area and four guide pulleys 32 in the lower area. The revolving means of transport 30 can be moved by a not depicted drive. It can be moved preferably in both directions. Every point of the revolving means of transport can therefore be brought in correspondence with an opening 32, which is meant for charging and discharging the incubator. Receptacles 35 for sample containers 20 are attached on the revolving means of transport, for example on the two chains 34. These have an angle 36 on the side, which is aligned upwards and in the top area is pivoted via on an axis 37 on the chain 34. As a result, the receptacles 35 can be aligned independent of how the chain 34 is momentarily running. Through the use of several vertical strands, the interior of the incubator can be reduced in its height, which can lead to a more uniform distribution of the temperature in its interior space.

FIG. 9 shows a detail of how the inlet opening 33 of the incubator of FIG. 7 can be designed. Many sample containers 20 are disposed beside one another on the receptacles 35, see FIG. 8. The incubator features a great length in the direction perpendicular to the drawing plane of FIG. 7. Since a high level of humidity of over 95 percent is and must be present in the incubator when charging and discharging sample containers, the inlet opening should only be opened as wide as necessary so that the humidify cannot sink. For this purpose, in a further development of the invention, a sluice 41 can be provided, as depicted in FIG. 9. In the opening, a reciprocating and rotatable cylinder 42 with a cut-out 43 is disposed. This cylinder 42 is rotatable about a horizontal axis 44. The wall 45 of the incubator above and below the opening 33 contains a wall section 46, which features the shape of a part of a circular cylinder. The rotatable cylinder 42 is disposed such that it lies with a sealing contact on the sections 46. In the depicted position, a sample container 20 can be inserted in the inferior of the cylinder 42. By rotating, the opening 43 it can then be aligned so that the cylinder can be pushed out. The interior of the cylinders 42 can also be partly filled out.

FIG. 10 shows the opening from the front side of the incubator. Since the opening can be very long due to the reasons already mentioned, it is possible to subdivide it. This is depicted in FIG. 10. Here the cylinder 42 with the cut-out 43 in the left position is half open, whilst it is closed in the following position. The subdivision of the opening in individual sluices can for example correspond to the width of a single sample container 20.

The FIGS. 11 and 12 now show a second embodiment of an incubator, how it can find application particularly in the unit according to the invention. Whereas with the incubator of FIGS. 7 and 8 the sample containers 20 are disposed in series along a path, another possibility of accommodating sample containers is proposed inside the incubator. Here individual parallel levels are provided for the sample containers 20, of which FIG. 11 only shows two levels, in each level, revolving belts 48 are provided, on whose fop strand 49 the sample containers 20 are disposed. The revolving belts 48 are guided around two guide pulleys 50 respectively.

In front of and behind the respective guide pulleys 50 of each level, two shafts are provided, in which a segment is respectively disposed, which is movable in height, as implied by the arrows 51. A transport segment 52 is disposed behind the inlet opening 33. This contains a conveyor belt 53, which is guided around two parallel guide pulleys 54. The width measured from left to right of this conveyor belt 53 in FIG. 11 approximately corresponds to the size of a sample container 20. Perpendicularly to the drawing plane of FIG. 11, the length of the conveyor belt 53 is as large as the entire dimension of the incubator in this direction. The transport segment 52, as already mentioned, can be moved over the entire height of the incubator. It contains a drive for its height movement, which is disposed outside the interior of the incubator as well as a drive for moving the conveyor belt around the guide pulleys 54, in both directions. A coupling element 55 is connected with the one guide pulley 54, which, with a corresponding counter coupling element 56 on the guide pulley 50 of the conveyor belt 48 of each level, together depict a coupling. When the transport segment 52 has been brought to the same height as a specific level, then the two coupling elements 55, 58 can be connected. Then the drive for the transport segment 52 can also drive the revolving belt 48 of the level.

On the opposite side of the inlet opening 33, facing away from the wall, a return segment 57 is disposed, which is practically made exactly like the transport segment 52. Here also, the drive for the guide pulley 54, which is facing the inferior of the incubator, contains a coupling element 55, which can interact with the corresponding coupling element 56 of the belt 48 of the level.

The coupling elements, which in FIG. 11 are only implied schematically, protrude through a partition 58 of the incubator in a room 59, which still lies within the hosing of the incubator, but is not part of the interior of the incubator. Between the gear wheels 60, which are disposed in the extension of the shafts of the guide pulleys 54, a coupling can be brought in engagement, which establishes the correct direction of rotation. This can be done with several gear wheels, wherein also other possibilities, for example a magnetic coupling can also find application.

The principle and/or the manner how the incubator according to FIGS. 11 of 12 can be operated is as follows. With the help of a transfer equipment movable along the length of the opening 33 available in front of the opening 33, a sample container is transferred through the opened sluice onto the transport segment 52 behind the opening 33. Products to be processed concurrently are pushed bit by bit onto the transport segment 52. As soon as no further sample containers for the process are provided, the transport segment 52 moves up to the height of a certain level. Then the coupling made of the coupling elements 55, 56 is connected and the horizontal drive of the transport segment 52 is put in motion. Thereby the series of sample containers 20 will be transferred from the transport segment 52 onto the belt 48 of the corresponding level, whilst the sample containers 20 available at the level are concurrently moved further by one cycle. Then the coupling is again released and the transport segment 52 is again moved in its starting position. This sequence is repeated several times. As soon as the sample containers 20 reach the opposite side at the end of the corresponding level, they will be taken over in the same manner from the returning transport segment 57. Also here a coupling operating in the same manner is provided. After taking over a series of sample containers 20, the return transport segment 57 brings them to another level where they are placed on the belt of this said level in a reverse manner. This belt then moves in the reverse direction, cycle-wise, so that the sample containers 20 at the end of the process can again be received by the transport segment 52 behind the inlet opening 33. From here, they can be removed from the incubator. The sample containers 20 are removed from the incubator in the same sequence in which they were put. Therefore, this involves a FIFO incubator or memory.

When the drive is coupled to the levels by the transport segments 52, 57, they work at the same speed everywhere, so that the number of levels which work in one direction is identical to the number of the levels which work in opposite direction. The levels that work in a given direction can be determined by means of the controls.

FIG. 13 now shows further equipment which is advantageous in connection with the unit according to the invention. It was already mentioned with reference to FIG. 1 that equipment for lifting the lid of sample containers is provided there, which however disturbs when processing the samples. On the other hand, the lid must again be placed on the right sample container, which means on the sample container from which it was removed. FIG. 13 now shows a second possibility of this. A rotating plate 61 is provided, which is rotatable about a perpendicular axis in the middle between the two strands of the transport device.

On the bottom side of the rotating plate 61 there are several receptacles 62 respectively disposed for a lid, in whose centre a suction cup 63 is provided, which is connected to negative pressure via a line 64. Under the receptacle for a sample container 20 a drive 65 is provided, which can push a plate 66 upwards. The drive 65 pushes the plate between the two arms of the receptacle and lifts the entire sample container 20 until the lid on the bottom side of the suction cup 63 comes to the unit. The controls can defect this through pressure drop, since no more air can now be sucked through the hose 64. As soon as this time point is reached, the drive 65 moves the plate 66 downwards again, so that the sample container 20 is again lowered onto the receptacle. The rotating plate 61 is then rotated so that the receptacle 62 with the lid now held firmly inside moves in the same cycle as the sample container 20 is moved. On the opposite side, the lid can again be put on the sample container 20, from which if was removed. Attention is therefore paid to ensure that the association between lids and sample containers is retained.

The invention therefore provides a unit to be operated automatically, with which a plurality of samples can be processed automatically. It can happen that the processing method can vary from sample to sample and that the processing time can be very short depending on the processing method and/or sample.

Inside the processing area, clean room conditions frequently prevail. The actuation activities which in particular means that the motion of manipulating devices or sample carriers must be adapted to the clean-room conditions, i.e., they must be minimized. The invention solves this in that it minimizes the number of manipulating devices without reducing the number of different processing possibilities. Furthermore, the invention limits the entire space required for processing the samples to a small area without reducing the throughput. Furthermore, the processing of a sample with different processing devices is enabled without moving the sample carriers between the processing steps.

Now regarding FIG. 14. In FIG. 1, at the end of the unit, it was illustrated on the processing station that all workplaces are disposed at a grip distance from the manipulating device 13. In a very schematic manner, FIG. 14 now shows that lateral processing stations 72 can be disposed on the extended disposition rail 71 for the processing stations with the manipulating device 13, on which activities can be carried out by workstations, which are independent of the manipulating device 13. Moreover, FIG. 14 shows that the entire processing area can be enclosed by an external wall 73. Also the transport section itself can run through a tunnel—not depicted—which is joined with the external wall 73 of the processing area.

Wherein the manipulating device 13 in the embodiment according to FIG. 1 and FIG. 14 is disposed at a fixed point so that the gripper arms can be rotated about a fixed perpendicular axis, FIG. 15 shows a modification in which the manipulating device 13 can be moved on a rail 74 in the longitudinal direction of the conveyor section. This makes it possible, not only to charge the processing stations on the disposition rail 71 but also on the linear processing stations 72.

FIG. 16 yet again shows a modified embodiment, where, in addition to the fixed manipulating device 13 on the base 105, also linear lateral processing stations 72 are disposed, on which feeding and other manipulating devices 75 are provided. These manipulating devices 75 can be moved along linear rails 76 and are therefore simple in design and easy to control. Of course more than one manipulating device 75 can be disposed on the rails 76. Due to a great number of individually controllable processing stations, also several piles 14 for lids with the corresponding grippers 15 can be provided in this embodiment.

Also in the embodiments according to FIG. 15 and FIG. 16, the bases 105 are disposed within the housing 73, which can be opened at the points where grasping is required. The housing is preferably made of a transparent material so that all processes can also be checked visually from outside.

The transport device, see FIG. 1, has revolving equipment, on which the receptacles for vessels or similar objects can be attached. According to the number of receptacles and if necessary also according the number of processes required for a certain receptacle, the revolving equipment can also have a remarkable length. In case linear equipment cannot be accommodated for certain reasons, then, according to the invention, also a disposition unit with several transport sections can be constructed, which can then be combined with one another so that delivery from one transport section to another transport section is possible. FIG. 17 shows an example. Furthermore, the invention determines how the transport section has to be constructed from grid dimension and individual sections of the transport section with the length corresponding to an integer multiple of the grid dimension. For example, in FIG. 17 a transport section 77 is equal to six-times the grid dimension in length and an adjoining transport section 78 is equal to eight-times the grid dimension in length.

With the disposition of FIG. 17, at the return pulley end of one transport section, a return pulley end of an adjoining transport section is disposed. In addition, a further transport section with its return pulley end can be disposed on a linear section of the first transport section.

With the illustration of FIG. 1, only the return pulley of the transport device is depicted and explained schematically. FIGS. 18 and 19 show a more detailed configuration in a somewhat larger detail. Here, the receptacle guide is separated from its drive. For the receptacles, transport nests 80 are provided, which are guided in conveyors 81 with an external slotted link 82 and an inner slotted link 83. The transport nests 80 are guided with castors 80 both on the outer as well as on the inner slotted link. As a result, not only is smooth running ensured, but also an exact positioning of the transport nests and the receptacles to be connected with them for the samples to be processed. The transport nests can be connected with a toothed belt 85 which then assumes the transporting task, but not the guiding task. The transport nests can mesh with a protrusion in a gap of the toothed belt 85. Thereby, the position of a transport nest in relation to the next transport nest can be set in a grid dimension that corresponds to the pitch of the toothed belt 85.

A toothed wheel 88 drives and returns the toothed belt 85, which features a form around its outer circumference which corresponds to the teeth of the toothed belt 85 but also has additional nests 87 for accommodating the transport nests, it is also considerable to accommodated transport nests relative to the plane of the drawing of FIG. 18 in another plane and to design the connection between the toothed belt 85 and the transport nest 80 such that also a toothed wheel can be used without such transport nests 87.

FIG. 19 shows a strongly simplified section of the manner in which a transport nest 80 engages in a guideway 81. This guideway 81 with the outer slotted link 82 and inner slotted link 83 is accommodated in a rail 88, under which a hollow profile 89 for accommodating supply and control lines is attached. On the top side of the transport nest 80 a receptacle 90 is inserted, which can be pushed both over as well as under the transport nest 80 with the help of a transfer device 91. For this purpose, the transfer device 91 possesses an arm 92 for example with a suction cup 93 on its free end. The transfer device 91 is attached to a console 93 which is laterally fixed on the rail 88. The transfer device can be attached anywhere on the rail 88, where a magazine 8, see FIG. 1, is located. Of course the transfer devices of this type can also be used to transfer receptacles from one transport device to another transport device, for example at the points, where this transport devices meet, see FIG. 17.

FIGS. 4 to 8 show a storage magazine 6, which is disposed beside a transport section and from which consumables can be discharged and placed on the transport section. FIGS. 20 and 21 now show a further embodiment of such a magazine, in which such consumables are stored. The consumables can involve for example microtitre plates in many different formats with or without a lid, or cell-culture bottles in different sizes. There can also be racks in microtitre plate format, which for example, are equipped with tubes. These products to be stored have a correct rack surface and a vertical wall which offers grasping surfaces for a cleaner. Moreover stackable products can be stacked on one another.

The magazine depicted in a short form in FIG. 20 contains a rack that features rollers 95 in the area associated with the end of its transport section, in order to be pushed over the floor 96. A table leg 97 is provided at a distance from the end at which the rollers 95 are mounted at the bottom. This fable leg 97 can be swivelled in. The rollers 95 carry a rack which is horizontal in the top part, where a transport device 98 in the form of a driven, conveyor belt is provided. Support plates 100 are disposed underneath the conveyor belt 99, which can be individually lifted by a lift unit 101 (not shown). The position of each support plate 100 supports a pile 102 for articles to be stored. On the right end, a receiving position is provided to manually place a pile 102.

A transport segment 103 is disposed between the transport device 98 with the support plates 100 and the transport device 3 accommodated in a tunnel 104, whose length is somewhat greater than the corresponding dimension of a pile 102 of articles. The transport segment 103 is movable within a limit in vertical direction, it contains a revolving conveyor belt that is also movable under control. The transport segment 103 serves the purpose of respectively transferring the lowest article of a pile 102 of articles through an opening in the housing 105 and an opening in the tunnel 104 to the transport device 3 of the processing device, for which the transfer equipment 91 of FIG. 19 is provided.

For further details on storage in magazines, reference is now drawn to FIG. 21. This shows a view of the disposition of FIG. 20, from left in FIG. 20. The piles 102 of stored articles are stored inside the magazine on the lowest edge flanges 106 of the side rail 107. This is implied for one article 108. If now a stack 102 of articles 108 should be transported further, then the support plates 100 are lifted with the help of the corresponding lift unit 101, so far until the pile lies on the conveyor belt 99, and lifted off the side flanges 106. Then transport can take place by means of the belts 99.

The transport segment 103 is mounted on a lift table 108, which can be lifted relative to an assembly rail 109 with the help of a drive. For this, the schematically implied guide rods 110 are provided. The lower end position of the lift table 108 and of the transport segment 103 is selected such that the lowest product in the pile just lies on the flange 106.

On the assembly rail there are stop mechanisms 111 pivoted respectively right and left, whose lower ends 112 can be moved by a horizontally acting elevating mechanism 113. The top ends 114 have catch plates 115 with which they can be brought to the unit on the side walls of the articles 108 and therefore they can hold such an article firmly. The top end position of the motion of the lift tables 108 is selected such that the second lowest article 108 of a pile lies on the transport segment 103 between the catch plates 115 of the stop mechanism 111.

The horizontally acting lift unit 113 can be magnets for example.

In FIG. 20, it is visible that an indexing device 120 is disposed respectively on the side of the magazine, next to the transport section 103, in the top and bottom areas. With the help of this indexing device, correct positioning and alignment of the magazine occurs relative to the tunnel 104 receiving the unit. Through an automatic mechanism, the magazine is utilized in the index position so that exact positioning is achieved for the product transfer to the base unit, in this position, the rollers 95 are lifted a bit from the floor 98, so that no mechanical over-definition occurs.

On the opposite side of the magazine, a control panel 121 is disposed, together with a grip 122. The control panel 121 is used to trigger the index device, which means, to release the magazine again.

The transport within the housing 105 of the magazine works as follows: Every transport segment, which means an area corresponding to a support plate 100, is fitted with a light-signal button which recognizes whether products are on the segment or not. The transport device conveys a product pile from one segment to the next when the transport segment in front of the pile is detected to be unoccupied. The support plates 100 of both segments are lifted so that an uninterruptible transport occurs from one to the next segment. As soon as the abandoned transport segment sees no more products, then the delivery is concluded successfully and both transport segments can again lower their support plates 100.

Should a product be transferred from the pile allocated to the transport segment 103, for transport to the base machine within the tunnel 104, this transport segment 103 will lift the pile so high until the second lowest product can be grasped and held by the system plates 115 of the stopper mechanism 111. The stopper mechanism is activated so that the system plates 115 can grasp. The transport segment 103 is now lowered whilst the pile is held by the stop mechanism 111. In the starting position, the object from the transfer equipment 31 now individually placed on the transport segment 103 can be held and transferred to the base unit.

Several such magazines described in FIG. 20 and 21 can be disposed along the transport section of the unit depicted in FIG. 1 and FIG. 17. After the index device releases, the magazines can be driven away and equipped anew outside the unit.

What is claimed is:

1. A unit for automated processing of biological samples, comprising:
a revolving transport device arranged for transporting sample containers,
at least one processing position for samples in the sample containers,
at least one processing device for the samples, and
a manipulating device operable to position the processing device opposite the processing position.

2. The unit according to claim 1, further comprising a magazine for storing consumable materials, and equipment for discharging the consumable materials and for delivering the consumable materials to the transport device.

3. The unit according to claim 1, further comprising an incubator for accommodating the samples in a defined environment.

4. The unit according to claim 1, wherein the processing position is disposed outside a transport section of the transport device.

5. The unit according to claim 1, further comprising transfer equipment operable for delivering the sample containers in at least one direction between the transport device and the processing position.

6. The unit according to claim 5, wherein the transfer equipment operates independent of the manipulating device.

7. The unit according to claim 1, wherein the manipulating device is configured for mechanical and functional coupling of the processing device.

8. The unit according to claim 1, further comprising a waiting station defining a waiting position disposed outside the processing position for the processing device.

9. The unit according to claim 8, wherein the waiting station is arranged such that the processing device can be cleaned, serviced and prepared for further work at said waiting station.

10. The unit according to claim 1, comprising at least one additional said processing device, and wherein the processing devices work independently of one another.

11. The unit according to claim 8, wherein at least one of said waiting position and said processing position is disposed such that the manipulating device can approach an operational position of the manipulating device through a linear motion along one degree of freedom of the manipulating device.

12. The unit according to claim 8, wherein at least two of one or more said waiting positions of one or more said processing devices and one or more said processing positions are disposed on a line parallel to a transport direction of the transport device.

13. The unit according to claim 8, wherein a degree of freedom of the manipulating device comprises a motion parallel to a transport direction of the transport device.

14. The unit according to claim 1, wherein the processing position is movable in a direction parallel to a transport direction of the transport device.

15. The unit according to claim 14, wherein the unit has plural processing stations associated with said one processing position.

16. The unit according to claim 1, wherein the transport device is arranged to move the sample containers along a path including two parallel strands with two return pulleys.

17. The unit according to claim 15, wherein at least one of the processing device, the processing position and the manipulating device is disposed in an area of one of the return pulleys.

18. The unit according to claim 1, wherein the processing position is disposed between transport sections of the transport device and a waiting position associated with the processing device.

19. The unit according to claim 1, wherein the sample containers comprise microtitre plates.

20. The unit according to claim 1, wherein the transport device comprises receptacles adapted for microtitre plates.

21. The unit according to claim 1, further comprising transfer equipment operable to transfer receptacles associated with the sample containers from the transport device to the processing position.

22. The unit according to claim 21, wherein the transfer equipment is configured to transfer microtitre plates from the transport device to the processing position.

23. The unit according to claim 2, wherein the magazine comprises at least one roller conveyor inclined for successive disposition of a series of sample containers.

24. The unit according to claim 23, wherein the roller conveyor charges the sample containers toward a front direction of the magazine against a stopper.

25. The unit according to claim 24, wherein the sample containers are charged by driven rollers.

26. The unit according to claim 24, wherein the sample containers are charged by driven rollers on which the sample containers lie.

27. The unit according to claim 25, wherein the sample containers are moved by a drive comprising a slip between a drive comprising at least one of a belt and a roller on the one hand, and the sample containers on the other hand.

28. The unit according to claim 25, wherein the sample containers are moved by a drive comprising a slip with at least one of a belt and a roller.

29. The unit according to claim 23, wherein a feed effect is generated by an inclined alignment of a guideway for the sample containers.

30. The unit according to claim 2, wherein the equipment for charging and discharging the consumable materials comprises roller conveyors.

31. The unit according to claim 2, wherein the equipment for charging and discharging the consumable materials comprises a surface coated with smooth-slide material.

32. The unit according to claim 23, wherein the roller conveyors comprise a sample-container guide disposed adjacent to the sample containers.

33. The unit according to claim 32, wherein a distance between a top side and a bottom side defined by a respective said sample-container guide is smaller than a sum of heights of a removed lid and a lower part of the sample container apart from the lid.

34. The unit according to claim 33, wherein the top side is defined at least partly by rollers of a respectively next higher roller conveyor of the magazine.

35. The unit according to claim 2, wherein the manipulating device is configured to lift a front sample container of a plurality of said sample containers above a stopper confining the sample containers.

36. The unit according to claim 2, wherein the manipulating device is configured to move a stopper from in front of a front sample container of a plurality of said sample containers, and into a horizontally opened gap in front of a next sample container for permitting the front sample container to move off.

37. The unit according to claim 3, wherein the incubator is arranged such that delivery of one of the sample containers into and out of the incubator occurs in a linear and horizontal manner.

38. The unit according to claim 37, wherein the incubator is designed such that movement of the sample container is caused by a manipulating device inside the incubator and a drive disposed outside the incubator.

39. The unit according to claim 37, wherein the incubator comprises an inlet sluice, which inlet sluice is only opened for placing and removing sample containers.

40. The unit according to claim 39, comprising conveyors associated with the incubator, the conveyors having a surface coated with smooth-slide material.

41. The unit according to claim 3, wherein the incubator comprises a plurality of receptacles for the sample containers, the receptacles being disposed along a closed path for transporting the sample containers in at least one of two opposite directions along said path.

42. The unit according to claim 41, wherein the receptacles are suspended on elongated flexible members of a paternoster lift.

43. The unit according to claim 42, wherein the elongated flexible members comprise multiple perpendicularly running strands.

44. The unit according to claim 37, wherein the incubator has a plurality of levels associated with drivable return belts.

45. The unit according to claim 44, further comprising a transport segment that is perpendicularly movable and stoppable at a height of any of the levels.

46. The unit according to claim 44, wherein a drive coupling is provided between a transport segment and a respective one of the drivable return belts for one of the levels and the drive coupling can be engaged when the transport segment is placed at a height corresponding to and is coupled to the level.

47. The unit according to claim 44, comprising a return transport segment that is perpendicularly movable and stoppable at a height of any of the levels.

48. The unit according to claim 47, wherein a drive coupling is provided between the return transport segment and a revolving belt of one of the levels, accessed when the return transport segment is at a height corresponding to the respective level and a drive of the return transport segment is coupled to the level.

49. The unit according to claim 47, comprising two said transport segments, respectively operable as transport and return transport segments and wherein the transport segments each can be driven in both directions.

50. The unit according to claim 3, wherein a manipulating device for feeding the incubator is provided outside the incubator and may be moved along an inlet opening of the incubator.

51. The unit according to claim 1, further comprising a loading station for manual input of the sample containers into the transport device.

52. The unit according to claim 1, further comprising an automatic ejection station for the sample containers.

53. The unit according to claim 1, further comprising an apparatus for removing and placing a lid from and onto one of the sample containers.

54. The unit according to claim 1, further comprising an additional processing station along a transport section of the transport device.

55. The unit according to claim 1, wherein the transport device is configured for transporting consumable material in receptacles provided for the sample containers.

56. The unit according to claim 2, wherein the magazine is configured for storing of consumable material and sample containers and is connectable with a transport section of the transport device.

57. The unit according to claim 56, wherein the magazine comprises a drive unit for step-wise further transport of piles of consumable material and transfer equipment for delivering a part of one of the piles to the transport device.

58. The unit according to claim 57, wherein the equipment of the magazine comprises transfer equipment that processes said one of the piles from bottom to top.

59. The unit according to claim 1, wherein the manipulating device is additionally movable in a longitudinal direction of the transport device.

60. The unit according to claim 1, wherein the transport device (1) comprises modular units that are connectable with one another.

61. The unit according to claim 2, wherein the consumable materials are stored in the magazine in said sample containers.
